(19) United States
(12) Patent Application Publication
(10) Pub. No.: US 2026/0325604 A1
(43) Pub. Date: Oct. 22, 2026

(54) THIOSTREPTON DOSING REGIMENS

(71) Applicant: RS Oncology, LLC, Cambridge, MA (US)

(72) Inventor: Brian Cunniff, Jericho, VT (US)

(21) Appl. No.: 19/106,704

(22) PCT Filed: Sep. 5, 2023
(86) PCT No.: PCT/US23/31983

§ 371 Date: Feb. 26, 2025

Related U.S. Application Data
(60) Provisional application No. 63/403,553, filed on Sep. 2, 2022.
(60) Provisional application No. 63/404,728, filed on Sep. 8, 2022.

(51) Int. Cl. A61K 38/16; A61P 35/00; A61P 35/04

(52) U.S. Cl. CPC A61K 38/164; A61P 35/00; A61P 35/04

(57) ABSTRACT

Disclosed herein are methods of administering thiostrepton, or pharmaceutical compositions thereof, as well as methods of treating cancer.

### CROSS REFERENCE TO RELATED APPLICATIONS

This application claims the benefit of priority to U.S. Provisional Patent Application No. 63/404,728, filed Sep. 8, 2022, and U.S. Provisional Patent Application No. 63/403,553, filed Sep. 2, 2022, each of which is hereby incorporated by reference in its entirety.

### BACKGROUND

Thiostrepton is a cyclic oligopeptide antibiotic that is also known by other names such as Bryamycin, Thiactin, alaninamide, HR4S203Y18, etc. Thiostrepton has the structure below:

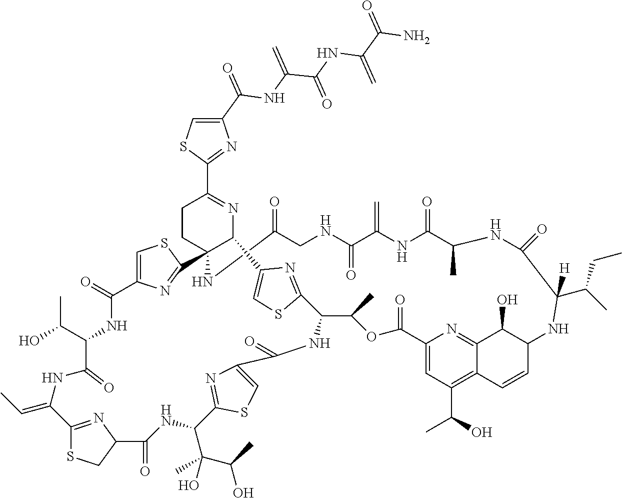 [SMILES: C=C(NC(=O)C(=C)NC(=O)c1csc(C2=N[C@@H]3c4csc(n4)[C@H]4NC(=O)c5csc(n5)[C@H]([C@](C)(O)[C@@H](C)O)NC(=O)C5CSC(=N5)/C(=C/C)NC(=O)[C@H]([C@@H](C)O)NC(=O)c5csc(n5)[C@]3(CC2)NC(=O)CNC(=O)C(=C)NC(=O)[C@H](C)NC(=O)[C@H]([C@@H](C)CC)NC2C=Cc3c([C@H](C)O)cc(nc3[C@H]2O)C(=O)O[C@@H]4C)n1)C(N)=O]

or a pharmaceutically acceptable salt thereof. Recent studies have shown that thiostrepton also has promising anticancer activity in addition to its antibiotic properties. There remains a need for safe and effective methods of administering thiostrepton for treating cancer.

### SUMMARY

In certain aspects, provided herein are methods of treating cancer, comprising administering to a subject in need thereof thiostrepton in a single dose of about 50 mg to about 500 mg once per week, thereby treating the cancer.

In certain aspects, provided herein are pharmaceutical compositions comprising thiostrepton and at least one pharmaceutically acceptable carrier, wherein the pharmaceutical composition comprises from about 50 mg to about 500 mg of thiostrepton.

In certain embodiments, the disclosure provides for administration of thiostrepton, or any of the pharmaceutical compositions comprising thiostrepton disclosed herein.

Also provided herein are methods of treating cancer, comprising administering to a subject in need thereof thiostrepton or a pharmaceutical composition comprising thiostrepton as described herein. In certain preferred embodiments, the cancer is malignant mesothelioma. In certain embodiments, thiostrepton is administered intraperitoneally, intrapleurally, subcutaneously, or intratumorally. In certain embodiments, thiostrepton is administered intraperitoneally, for example, using an indwelling intraperitoneal catheter (IPC) or a drainage port catheter. In certain embodiments, the methods further comprise removing liquid from a body cavity (such as a pleural effusion or asities fluid from the peritoneal cavity), for example, to dryness, before administering thiostrepton. In some embodiments, the thiostrepton is administered once per week for at least 3 weeks. In preferred embodiments, a single dose of thiostrepton is administered once per week at about 90 mg to about 450 mg, such as about 90 mg, about 180 mg, about 270 mg, about 360 mg, and about 450 mg.

### BRIEF DESCRIPTION OF THE DRAWINGS

FIG. 1A depicts the nucleus and mitochondria of normal mesothelial cells.

FIG. 1B depicts the nucleus and mitochondria of malignant mesothelial cells.

FIG. 2A depicts the EC₅₀ of thiostrepton in normal mesothelial and various mesothelioma cell lines (varying BAP1 expression).

FIG. 2B depicts PRX3 knock down with siRNA significantly reduces MM (malignant melanoma, HM cell line-pleural biphasic) proliferation (red squares). Co-expression of the H₂O₂ scavenger catalase (CAT, green triangles) or mitochondrial targeted catalase (mCAT, blue inverted triangles) rescues proliferation in cells lacking PRX3 expression.

FIG. 2C depicts the weight of residual tumours (grams) resected from mice harboring MM xenografts in the peritoneal cavity following four weeks of treatment with 20 mg/ml of a thiostrepton composition 2× weekly. ** p<0.01

FIG. 3A depicts malignant pleural effusions (MPE) collected from patients with metastatic disease.

FIG. 3B depicts adherent tumour spheroids grown in MPE supernatant.

FIG. 3C depicts non-adherent immune cells grown in MPE supernatant.

FIG. 3D depicts relative PRX3 inactivation by thiostrepton in both tumour (adherent) and immune (non-adherent) cells.

FIG. 3E indicates MPE derived tumour cells are equally sensitive to thiostrepton compared to established MM cell lines.

FIG. 4A depicts draining the plural effusion to dryness.

FIG. 4B depicts administration of thiostrepton using an indwelling intraperitoneal catheter (IPC).

FIG. 4C depicts securing the IPC on the patient.

FIG. 5 depicts a summary of the thiostrepton dose escalation study.

FIG. 6 depicts an exemplary Phase 1 dose escalation trial for treating cancer with compounds and/or compositions of the disclosure.

FIG. 7 depicts an exemplary Phase 1 dose expansion trial at RP2D, TS indicating a compound or composition of the disclosure.

FIG. 8 is a Swimmer's plot of three exemplary patient outcomes from the Phase 1/2 MITOPE trial. BOR indicates best overall response, PR indicates partial reduction of disease, and SD indicates stable disease.

FIG. 9 shows a treatment overview for Patient 1.

FIG. 10 is a chart depicting a decrease in pleural effusion drainage in Patient 1 as a function of time.

FIGS. 11A-11C show cross-sectional CT scans of the CP node of Patient 1 at pre-treatment (A), 6 weeks on treatment (B), and 12 weeks on treatment (C). The scans confirm partial response to treatment.

FIGS. 12A-12C show cross-sectional CT scans of the right mediastinal pleural nodule of Patient 1 at pre-treatment (A), 6 weeks on treatment (B), and 12 weeks on treatment (C). The scans confirm partial response to treatment.

FIG. 13 shows a treatment overview for Patient 2.

FIGS. 14A-14C show cross-sectional CT scans of the right pleural node of Patient 2 at pre-treatment (A), 6 weeks on treatment (B), and 12 weeks on treatment (C). The scans confirm stable disease.

FIGS. 15A-15C show cross-sectional CT scans of the GB fundus lesion of Patient 2 at pre-treatment (A), 6 weeks on treatment (B), and 12 weeks on treatment (C). The scans confirm stable disease.

FIG. 16 is a chart depicting a decrease in pleural effusion drainage in Patient 2 as a function of time.

FIG. 17 shows a treatment overview for Patient 3.

FIG. 18 is a chart depicting a decrease in pleural effusion drainage in Patient 3 as a function of time.

FIGS. 19A-19C show cross-sectional CT scans of the tumor of Patient 3 at pre-treatment (A), 6 weeks on treatment (B), and 12 weeks on treatment (C). The scans confirm stable disease up to 12 weeks on treatment.

FIG. 20 shows a cross-sectional CT scan of new liver metastases in Patient 3, which indicates disease progression at the 12 week scan.

FIG. 21 shows a CT scan of new lytic bone metastases in Patient 3, which indicates disease progression at the 12 week scan.

### DETAILED DESCRIPTION

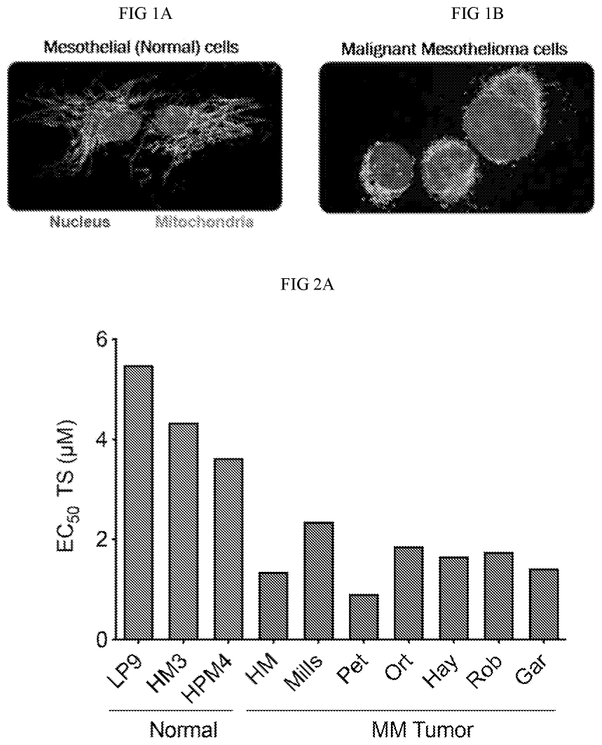

Tumour cells generate elevated levels of reactive oxygen species (ROS) and therefore exhibit increased expression and activity of critical ROS scavenging pathways, including the mitochondrial peroxide scavenging enzyme peroxiredoxin 3 (PRX3). PRX3 is a peroxidase responsible for metabolizing ~90% of mitochondrial ROS, primarily H₂O₂. PRX3 transcript levels are upregulated, compared to normal tissues, in approximately 50% of cancers (data from the GEPIA2 database). Genetic knock down of PRX3 in human tumour cells results in sensitization to apoptosis. The mitochondria of malignant mesothelioma (MM) cells are structurally and functionally altered leading to disrupted metabolic function that supports tumour growth and can be therapeutically targeted (see FIG. 1A, 1B).

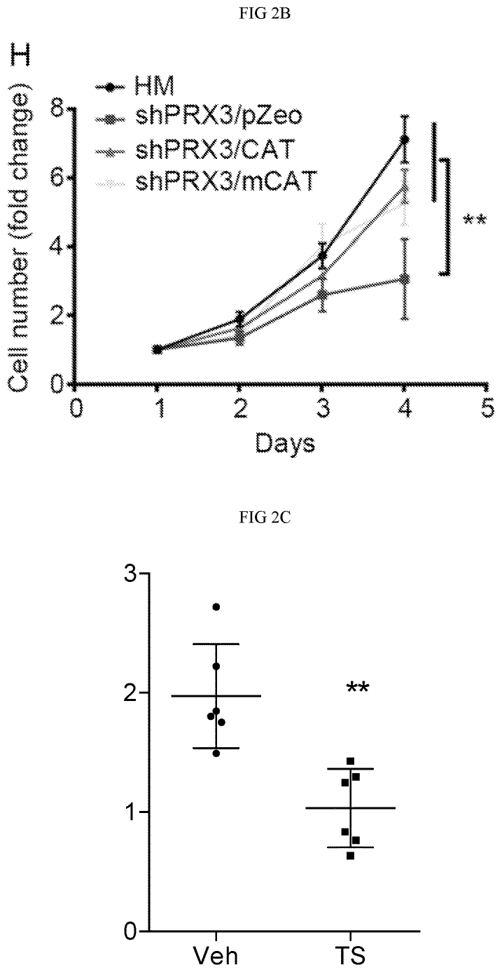

Thiostrepton is a covalent inhibitor that inactivates PRX3 peroxidase activity through direct adduction of active site cysteine residues, in turn, inducing oxidative stress to levels incompatible with tumour cell survival. The EC₅₀ of thiostrepton in normal mesothelial and various mesothelioma cell lines with varying BAP1 expression is shown in FIG. 2A. PRX3 knock down with siRNA significantly reduces MM (HM cell line-pleural biphasic) proliferation as depicted in FIG. 2B. Co-expression of the H₂O₂ scavenger catalase rescues proliferation in cells lacking PRX3 expression. The weight of the tumor is significantly smaller than that of a control in residual tumours resected from mice harboring MM xenografts in the peritoneal cavity following four weeks of treatment with 20 mg/ml of a thiostrepton composition 2× weekly (FIG. 2C), thereby demonstrating the preclinical rationale for thiostrepton's biological activity.

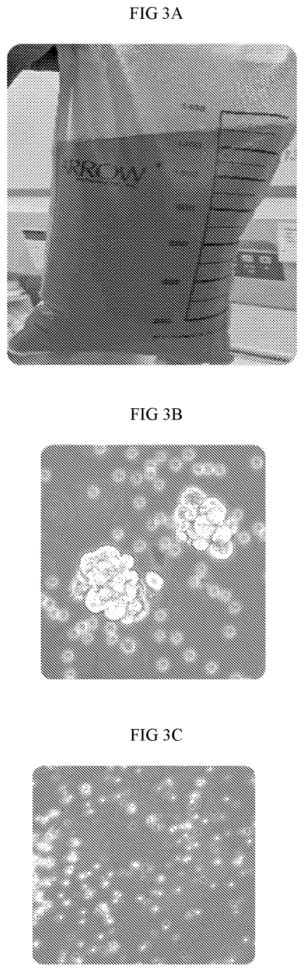

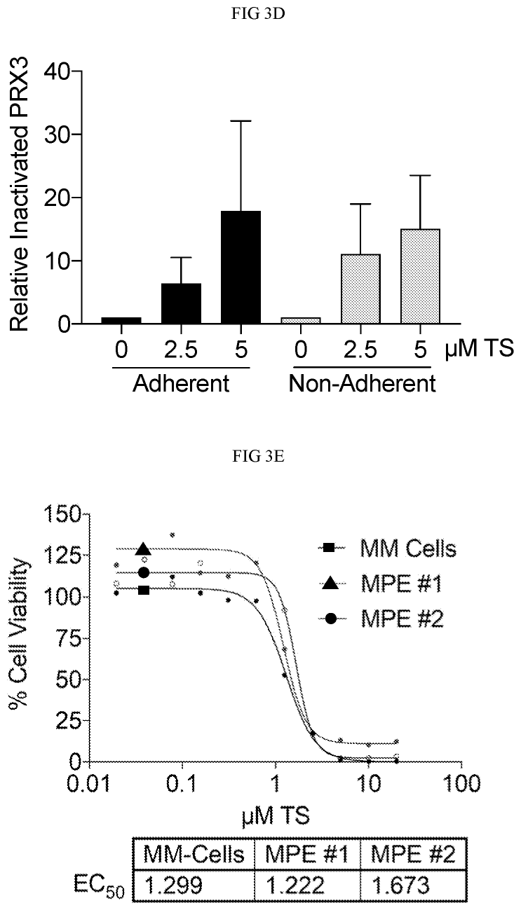

Small amounts of pleural effusion in the pleural space is physiologically normal. Mesothelioma and metastatic disease to the lungs often results in build-up of excess fluid (~15% of cancers). Malignant pleural effusion (MPE) is routinely drained using an intrapleural catheter. Thiostrepton retains activity in patient-derived malignant pleural effusions (MPE). MPE contains tumour and immune cells and allows for evaluation of the efficacy of thiostrepton in this disease, where both cell types are inhibited by thiostrepton. Specifically, adherent tumour spheroids and non-adherent immune cells can be grown in a medium containing MPE supernatant. (FIGS. 3A, 3B, 3C). PRX3 is inactivated by thiostrepton in both tumour (adherent) and immune (nonadherent) cells (FIG. 3D). Moreover, MPE derived tumour cells are equally sensitive to thiostrepton compared to established MM cell lines as shown in FIG. 3E.

In certain aspects, provided herein are methods of treating cancer, comprising administering to a subject in need thereof thiostrepton in a single dose of about 50 mg to about 500 mg once per week, thereby treating the cancer.

In certain embodiments, the single dose is from about 90 mg to about 450 mg. In some such embodiments, the single dose is selected from about 90 mg, about 120 mg, about 180 mg, about 270 mg, about 360 mg, and about 450 mg.

In some embodiments, the single dose is administered to the subject once per week for at least 3 weeks. In certain embodiments, the single dose in one week is the same amount as the single dose administered in a different week. In various embodiments, first single dose is administered for 3 weeks, followed by administration of a second single dose for the next 3 weeks, wherein the first single dose and the second single dose are different. In certain such embodiments, the second single dose is greater than the first single dose. In other embodiments, the second single dose is less than the first single dose.

In certain embodiments, the thiostrepton is present in a pharmaceutical composition further comprising at least one pharmaceutically acceptable carrier.

In some embodiments, the thiostrepton is administered locally. In certain embodiments, the thiostrepton is administered intraperitnoeally, intrapleurally, subcutaneously, or intratumorally.

In certain embodiments, the thiostrepton is administered by a catheter, a tube, or a needle. In some embodiments, the thiostrepton is administered by an indwelling intraperitoneal catheter (IPC).

In some embodiments, the cancer is selected from lung, breast, prostate, melanoma, esophageal, leukemia, cervical, liver, colon, gastric, colorectal, glioblastoma, head and neck, pancreatic, mesothelioma, and ovarian. In further embodiments, the cancer is malignant mesothelioma. In yet further embodiments, the cancer is malignant pleural mesothelioma. In still further embodiments, the cancer is malignant epitheloid pleural mesothelioma. In other embodiments, the cancer is malignant peritoneal mesothelioma. In certain embodiments, the cancer is lung cancer. In certain such embodiments, the lung cancer is an adenocarcinoma. In some embodiments, the cancer is metastatic.

In certain embodiments, the subject experiences stable disease for at least 12 weeks after administration of a first dose of thiostrepton. In some such embodiments, the subject experiences stable disease for at least about 12 weeks to about 52 weeks after administration of a first dose of thiostrepton. In some embodiments, the subject experiences stable disease for at least 18 weeks after administration of a first dose of thiostrepton. In certain such embodiments, the subject experiences stable disease for at least about 18 weeks to about 52 weeks after administration of a first dose of thiostrepton. In certain embodiments, the subject experiences stable disease for at least 24 weeks after administration of a first dose of thiostrepton. In some such embodiments, the subject experiences stable disease for at least about 24 weeks to about 52 weeks after administration of a first dose of thiostrepton.

In some embodiments, the subject experiences progression free survival for at least 12 weeks after administration of a first dose of thiostrepton. In some such embodiments, the subject experiences progression free survival for at least about 12 weeks to about 52 weeks after administration of a first dose of thiostrepton. In some embodiments, the subject experiences progression free survival for at least 18 weeks after administration of a first dose of thiostrepton. In certain such embodiments, the subject experiences progression free survival for at least about 18 weeks to about 52 weeks after administration of a first dose of thiostrepton. In certain embodiments, the subject experiences progression free survival for at least 24 weeks after administration of a first dose of thiostrepton. In some such embodiments, the subject experiences progression free survival for at least about 24 weeks to about 52 weeks after administration of a first dose of thiostrepton.

In certain embodiments, the subject experiences partial response for at least 12 weeks after administration of a first dose of thiostrepton. In some such embodiments, the subject experiences partial response for at least about 12 weeks to about 52 weeks after administration of a first dose of thiostrepton. In some embodiments, the subject experiences partial response for at least 18 weeks after administration of a first dose of thiostrepton. In certain such embodiments, the subject experiences partial response for at least about 18 weeks to about 52 weeks after administration of a first dose of thiostrepton. In certain embodiments, the subject experiences partial response for at least 24 weeks after administration of a first dose of thiostrepton. In some such embodiments, the subject experiences partial response for at least about 24 weeks to about 52 weeks after administration of a first dose of thiostrepton.

In certain embodiments, the partial response comprises a reduction in tumor size of at least 10% relative to the tumor size before administration of thiostrepton. In further embodiments, the partial response comprises a reduction in tumor size of at least 30% relative to the tumor size before administration of thiostrepton. In yet further embodiments, the partial response comprises a reduction in tumor size of at least 60% relative to the tumor size before administration of thiostrepton.

In certain embodiments, the subject experiences pleural effusion. In some such embodiments, the volume of pleural effusion is reduced 10 weeks after administration of a first does of thiostrepton relative to the volume of pleural effusion before administration of thiostrepton. In certain embodiments, the volume of pleural effusion is reduced 5 weeks after administration of a first does of thiostrepton relative to the volume of pleural effusion before administration of thiostrepton. In some embodiments, the volume of pleural effusion is reduced 1 week after administration of a first does of thiostrepton relative to the volume of pleural effusion before administration of thiostrepton.

In certain embodiments, the volume of pleural effusion is about 30% less than the volume of pleural effusion before administration of thiostrepton. In some embodiments, the volume of pleural effusion is about 60% less than the volume of pleural effusion before administration of thiostrepton. In certain embodiments, the volume of pleural effusion is about 90% less than the volume of pleural effusion before administration of thiostrepton.

In certain aspects, provided herein are pharmaceutical compositions comprising thiostrepton and at least one pharmaceutically acceptable carrier, wherein the pharmaceutical composition comprises from about 50 mg to about 500 mg of thiostrepton.

In certain embodiments, the composition comprises from about 90 mg to about 450 mg of thiostrepton. In some embodiments, the composition comprises about 90 mg, about 180 mg, about 270 mg, about 360 mg, or about 450 mg of thiostrepton.

The compositions and methods described herein may be utilized to treat an individual in need thereof. The term “pharmaceutical composition” means a composition that comprises thiostrepton and at least one pharmaceutically acceptable carrier. The terms “active compound” and “active ingredient” refer to thiostrepton. In certain embodiments, the individual is a mammal such as a human, or a non-human mammal. In certain preferred embodiments, the subject or the mammal is a human. When administered to an animal, such as a human, the composition or the compound is preferably administered as a pharmaceutical composition comprising, for example, an active compound described herein and a pharmaceutically acceptable carrier. Pharmaceutically acceptable carriers are well-known in the art and include, as a non-limiting example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of an active compound such as an active compound described herein. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a compound described herein. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, intraperitoneally; intrapleurally; subcutaneously; intratumorally; orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); transdermally (for example as a patch applied to the skin); and topically (for example, as a cream, ointment or spray applied to the skin). The compound may also be formulated for inhalation. In some embodiments, the pharmaceutical composition is administered locally. In some embodiments, the pharmaceutical composition is administered by a catheter, a tube, or a needle. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the compound that produces a therapeutic effect.

Methods of preparing these formulations or compositions include the step of bringing into association an active compound, such as thiostrepton, with the carrier and, optionally, one or more accessory ingredients. In general, the formulations are prepared by uniformly and intimately bringing into association a compound described herein with liquid carriers, or finely divided solid carriers, or both, and then, if necessary, shaping the product.

Liquid dosage forms include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the liquid compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

The phrases “parenteral administration” and “administered parenterally” as used herein means modes of administration other than enteral and topical administration, usually by injection, and includes, without limitation, intraperitoneal, intrapleural, subcutaneous, intratumoral, intravenous, intramuscular, intraarterial, intrathecal, intracapsular, intraorbital, intracardiac, intradermal, transtracheal, subcuticular, intraarticular, subcapsular, subarachnoid, intraspinal and intrasternal injection and infusion. In some embodiments, the pharmaceutical composition is administered intraperitoneally, intrapleurally, subcutaneously, or intratumorally. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions described herein include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug after injection, for example, subcutaneous injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods described herein, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors that influence the effective amount may include, but are not limited to, the severity of the patient's condition, the disorder being treated, the stability of the compound, and, if desired, another type of therapeutic agent being administered with the compound described herein. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable dose of an active compound used in the compositions and methods described herein will be that amount of the compound that is the lowest dose effective to produce a therapeutic effect. Such an effective dose will generally depend upon the factors described above.

If desired, the effective dose of the active compound may be administered as one, two, three, four, five, six or more sub-doses administered separately at appropriate intervals, optionally, in unit dosage forms. In some embodiments, thiostrepton is administered once a week. In certain embodiments, thiostrepton is administered for two, three, four, five or six consecutive weeks. In some embodiments, thiostrepton is administered every other week, once every three weeks, or once every four weeks at a rate of once or twice each week.

The patient receiving this treatment is any animal in need, including primates, in particular humans; and other mammals such as equines, cattle, swine, sheep, cats, and dogs; poultry; and pets in general.

In certain embodiments, compounds described herein may be used alone or conjointly administered with another type of therapeutic agent.

The present disclosure includes the use of pharmaceutically acceptable salts of compounds described herein in the compositions and methods described herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn or other metal salts. In certain embodiments, contemplated salts include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, l-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d glucoheptonic acid, d gluconic acid, d glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, l-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, propionic acid, 1-pyroglutamic acid, salicylic acid, sebacic acid, stearic acid, succinic acid, sulfuric acid, 1 tartaric acid, thiocyanic acid, p-toluenesulfonic acid, trifluoroacetic acid, and undecylenic acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification.

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats). A subject may be male or female. In some embodiments, the subject is greater than 18 years old. In certain embodiments, a subject preferably has an ECOG (Eastern Cooperative Oncology Group) score of 0-1. In certain embodiments, a patient may have a histological diagnosis of MPE caused by non-mesothelioma solid tumour or mesothelioma. In some embodiments, the patient has received at least one prior standard of care treatment regimen, with documented progression and no approved alternative available. In some embodiments, the patient has resolution of all acute reversible toxic effects of prior therapy to Grade ≤1. In certain embodiments, the patient has a paraffin block of his or her most recent biopsy. In some embodiments, the patient has adequate organ function as defined by lab values before administration of thiostrepton. In some embodiments, the subject is postmenopausal, surgically sterile, or using effective birth control.

In some embodiments, the patient has not have prior systemic anti-cancer or radiation therapy before administration of thiostrepton. In some embodiments, the patient has not have surgery within 3 weeks or within 5 half-lives before administration of thiostrepton. In some embodiments, the patient has not had treatment with an investigational product/device within 30 days before administration of thiostrepton. In certain embodiments, the patient has not had a previous malignancy other than the cancer to be treated before administration of thiostrepton. In certain embodiments, the patient does not have tumours or loculations that would render intrapleural administration incomplete or ineffective. In certain embodiments, the patient does not have a known hypersensitivity to thiostrepton or a pharmaceutical composition excipient. In some embodiments, the patient does not any surgical or medical condition that is likely to interfere with thiostrepton treatment. In some embodiments, the patient does not have human immunodeficiency virus (HIV) or active infection with hepatitis B; or hepatitis C in absence of a sustained virologic response. In certain embodiments, the patient is not pregnant or breast-feeding. In some embodiments, the patient does not have a symptomatic or unstable CNS tumour or metastases or carcinomatous meningitis. In some embodiments, the patient has not used systemic corticosteroids within 15 days before administration of thiostrepton or other immunosuppressive drugs within 3 weeks before administration of thiostrepton.

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered intraperitoneally, intrapleurally, subcutaneously, intratumorally, intravenously, arterially, intradermally, intramuscularly, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

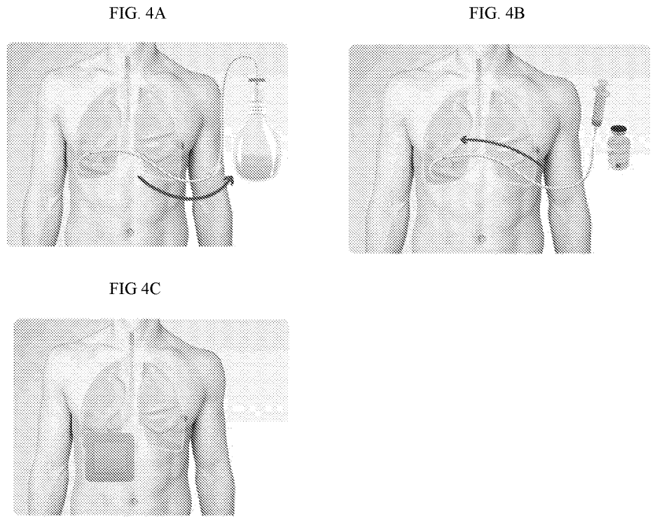

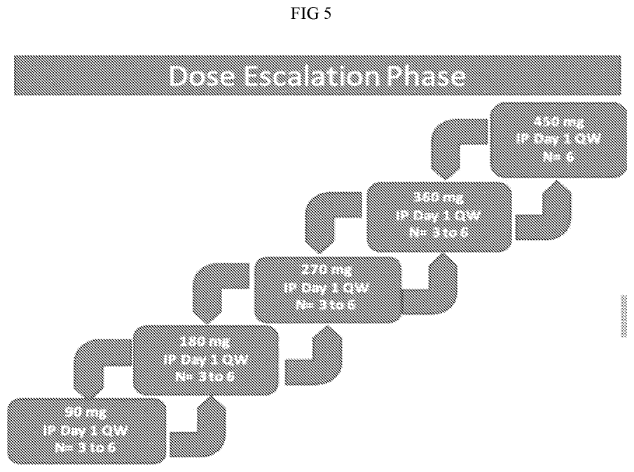

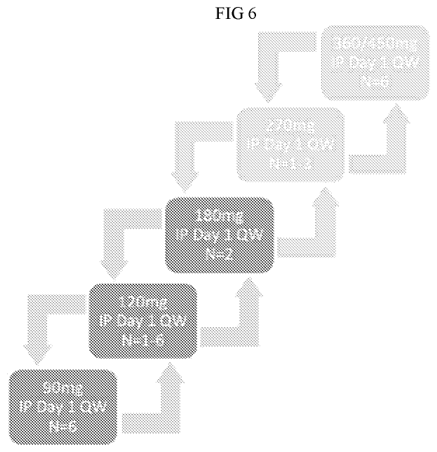

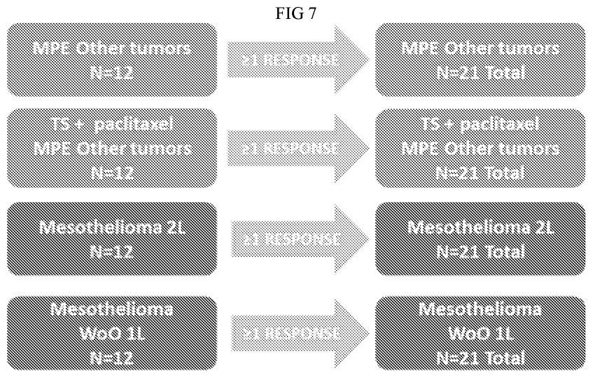

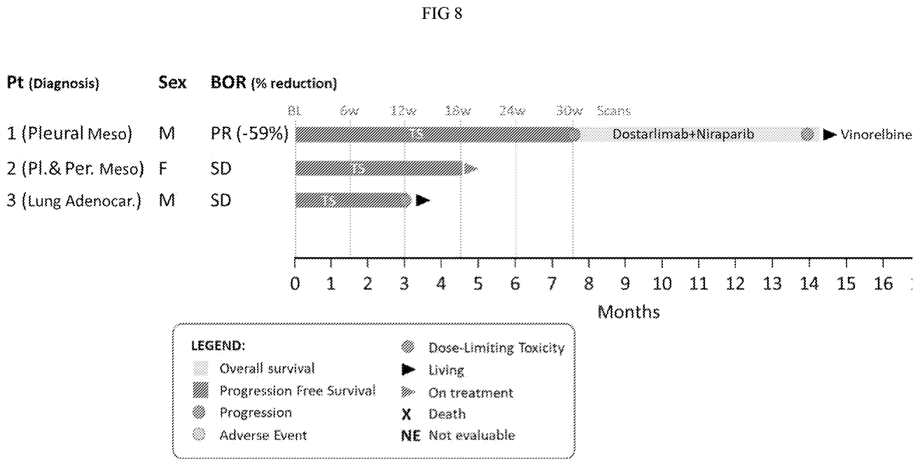

In certain embodiments, administration of thiostrepton or any of the pharmaceutical compositions comprising thiostrepton disclosed herein can be carried out using an indwelling intraperitoneal catheter (IPC). In some embodiments, administration occurs once a week or twice a week, preferably once a week. In some embodiments, the methods further comprise removing liquid from a pleural effusion, for example, before administration of thiostrepton (FIG. 4A). In certain embodiments, after administration of thiostrepton (FIG. 4B), the IPC is secured until the next dosing time point (FIG. 4C).

In certain embodiments, the single dose amount of thiostrepton ranges from about 50 mg to about 500 mg. In some embodiments, the single dose amount of thiostrepton ranges from about 90 mg to about 450 mg. In preferred embodiments, the single dose is selected from about 90 mg, about 180 mg, about 270 mg, about 360 mg, and about 450 mg. In some embodiments, the single dose is administered to the subject once per week, for example, for at least 3 weeks.

In some embodiments, the single dose of thiostrepton may be increased every three week period, for example, such that the patient at weeks 1-3 is dosed at 90 mg once each week, then optionally at weeks 4-6 is dosed at 180 mg once each week, then optionally at weeks 7-9 is dosed at 270 mg once each week, then optionally at weeks 10-12 is dosed at 360 mg once each week, and finally optionally at weeks 13-15 is dosed at 450 mg once each week. Each patient may complete any or all of the 3 week sessions (FIG. 5). In some embodiments, the dosing regimen may be paused, halted, or the patient may move to a lower dose, for example, in the event of toxicity or an adverse event.

In some embodiments, the methods further comprise obtaining a tumour biopsy from the patient before administering thiostrepton. In some embodiments, the methods further comprise obtaining a tumour biopsy after administration of the third dose of thiostrepton.

In other embodiments, the pharmaceutical composition comprises from about 5 mg thiostrepton/mL to about 50 mg thiostrepton/mL. In some embodiments, the pharmaceutical composition comprises about 10 mg thiostrepton/mL, about 20 mg thiostrepton/mL, about 30 mg thiostrepton/mL, about 40 mg thiostrepton/mL and about 50 mg thiostrepton/mL. In a preferred embodiment, the pharmaceutical composition comprises 20 mg thiostrepton/mL.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., when at least 5% of drug product is detectable systemically with industry acceptable methodology, or when the two agents are simultaneously effective in the patient, which may include synergistic effects of the two agents). For example, the different therapeutic compounds can be administered either in the same formulation or in separate formulations, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic agents.

In some embodiments, a first single dose in one week is the same amount as the single dose administered in a different weeks. In certain embodiments, a first single dose is administered for 3 weeks, followed by administration of a second single dose for the next 3 weeks, wherein the first single dose and the second single dose are different. In some embodiments, the second single dose is greater than the first single dose. In other embodiments, the second single dose is less than the first single dose.

A “therapeutically effective amount” or a “therapeutically effective dose” of a compound or other agent described herein is an amount of a drug or an agent that, when administered to a subject will have the intended therapeutic effect. The full therapeutic effect does not necessarily occur by administration of one dose of such a drug or agent, and may occur only after administration of a series of doses (multiple consecutive doses). Thus, a therapeutically effective amount may be administered in one or more administrations. The precise effective amount needed for a subject will depend upon, for example, the subject's size, health and age, and the nature and extent of the condition being treated, such as cancer.

As used herein, the terms “optional” or “optionally” mean that the subsequently described event or circumstance may occur or may not occur, and that the description includes instances where the event or circumstance occurs as well as instances in which it does not.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In certain embodiments, disclosed herein are methods of treating a cancer (e.g., solid tumor or hematological cancer) comprising administering to a subject in need thereof a therapeutically effective amount of any of the compounds described herein, or a composition of that compound. In certain embodiments, the cancer (solid tumor or hematological) is selected from lung, breast, prostate, melanoma, esophageal, leukemia, cervical, liver, colon, gastric, colorectal, glioblastoma, head and neck, pancreatic, mesothelioma, and ovarian. In certain embodiments, the cancer is selected from mesothelioma, lung, ovarian, and breast. In some embodiments, the cancer is malignant mesothelioma.

### EXAMPLES

### Exemplary Phase 1/2 Dose-Escalation and Expansion Study

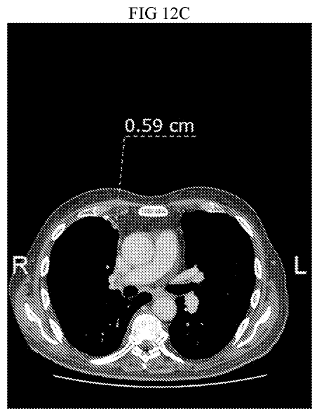

A Phase 1/2 Dose-Escalation and Expansion Study was carried out to determine safety, tolerability, and recommended Phase 2 dose of exemplary thiostrepton compositions in patients with malignant pleural effusion due to advanced/metastatic solid tumors including mesothelioma. Preliminary results for three patients (Patient 1, Patient 2, and Patient 3) are provided herein. FIGS. 6-20 provide overviews of the trial, treatment details, and exemplary preliminary data for these three patients.

The terms Partial Response (“PR”), Stable Disease (“SD”), Best Overall Response (“BOR”) are used in accordance with the definitions and criteria set forth in the RECIST guidelines, version 1.1 (Eur. J. Cancer. 2009, 45, 228-247).

### Patient 1:

84 years old male, ECOG 1
Background Medical history: Hypertension, Hypercholesterolaemia, Benign Prostate Hypertrophy
Diagnosed in December 2020 with Malignant Epithelioid Pleural Mesothelioma
Prior first line (1L) treatment: Carboplatin+pemetrexed BOR=Stable Disease
Patient progressed on 1L treatment
Treated at 90 mg dosage of thiostrepton, administered once weekly

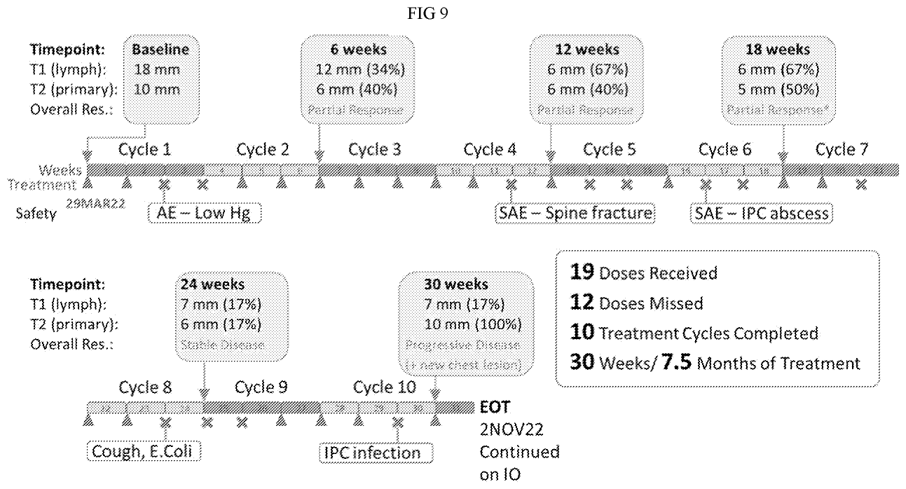

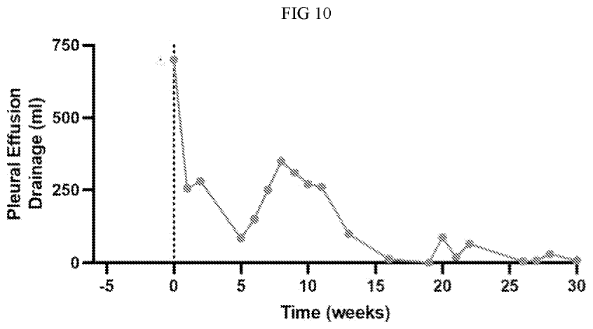

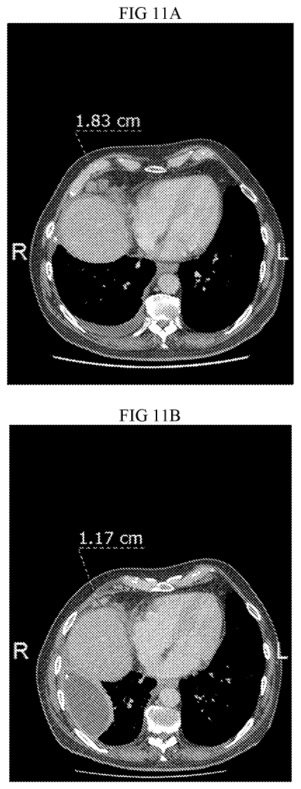

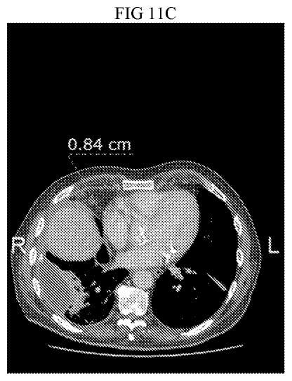

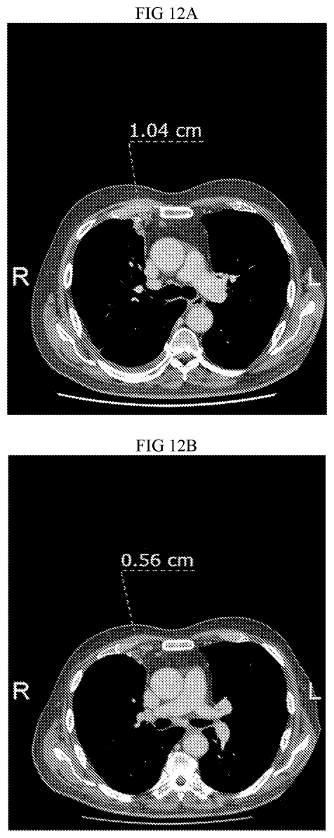

As shown in the chart in FIG. 9, Patient 1 showed Partial Response (reduction in tumor size of about 59%) for 18 weeks after initial thiostrepton treatment, and Stable Disease through week 24. FIG. 10 shows that Patient 1 experienced a significant decrease in pleural effusion through 30 weeks of thiostrepton treatment. FIGS. 11A-11C are CT scans of Patient 1 at pre-treatment (11A), 6 weeks on treatment (11B), and 12 weeks on treatment (11C).

### Patient 2:

75 years old female, ECOG 1
Diagnosed on 12 Apr. 2019 with Malignant Epithelioid pleural mesothelioma with infradiaphragmatic disease.
Background Medical history: Hypertension, Prior TB −25 years ago complicated by bronchiectasis.
1st L: chemo Carboplatin+Pemetrexed BOR=Stable Disease
2^{nd} L: Vinorelbine; BOR=PD
3^{rd} L: Nivolumab; BOR=SD
4^{rd} L: Gemcitabine; BOR=PD
5^{th} L: Pem/Carbo (re-challenge); BOR=PR
Patient progressed on rechallenge Carb/Pem
Treated at 90 mg thiostrepton dosage, administered once weekly

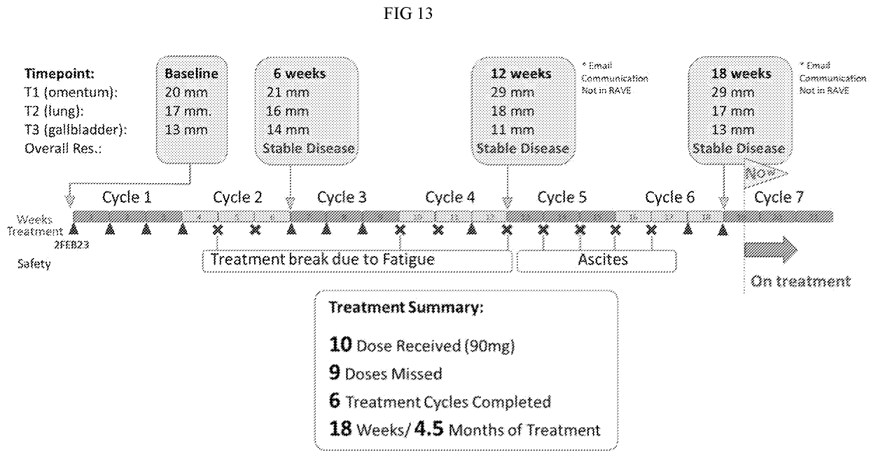

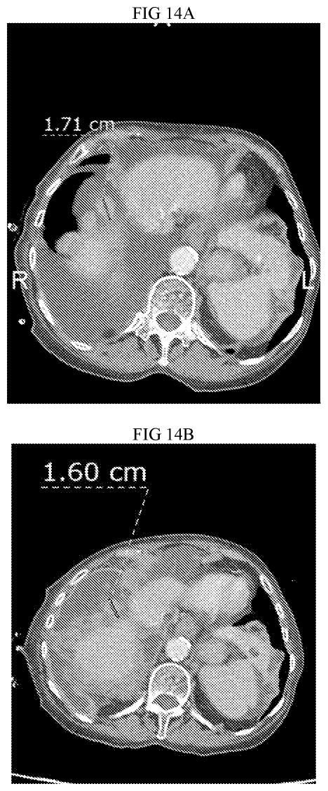

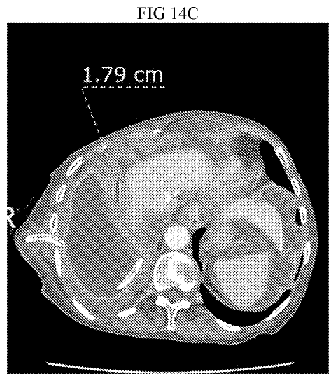

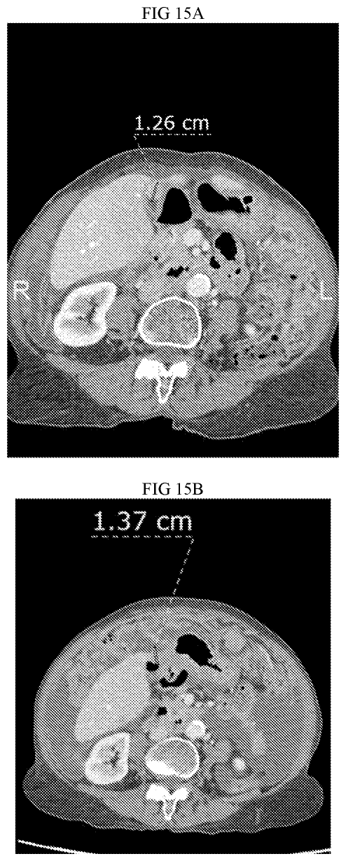

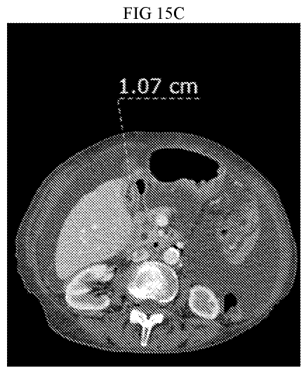

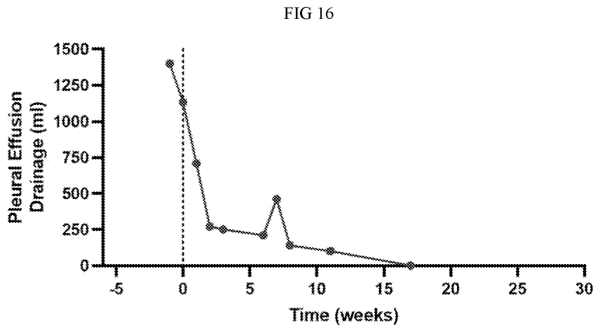

As shown in FIG. 13, Patient 2 showed stable disease through at least 18 weeks of treatment. FIG. 16 shows that Patient 2 experienced a reduction in pleural effusion drainage through 18 weeks of treatment. FIGS. 14A-14C and FIGS. 15A-15C are CT scans of Patient 2 at pre-treatment (14A, 15A), 6 weeks on treatment (14B and 15B), and 12 weeks on treatment (14C and 15C).

### Patient 3:

74 years old male, ECOG 1
Diagnosed on 17 Jul. 2021 with T4N2M1a adenocarcinoma of Lung
Background Medical history: Glaucoma, Atrial flutter—treated with ablation.
1^{st} line: Carboplatin+Pemetrexed+Pembro; BOR=Stable Disease
Patient progressed on 25^{th} February 23
Developed significant pleural effusion—MPE
IPC inserted 9^{th} March 23—drained large volumes of haemorrhagic pleural effusion in excess of 4 litres per week.
Initiated trial treatment on 17MAR23
Treated at 90 mg thiostrepton dosage, administered once weekly

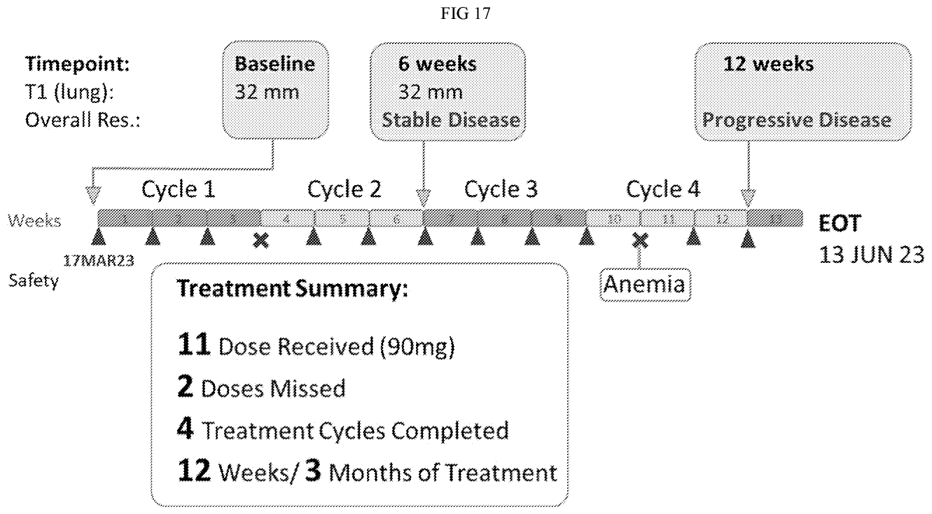

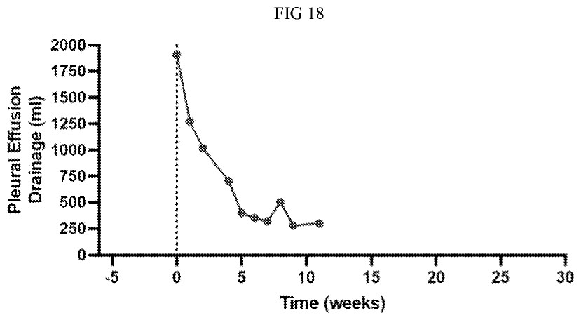

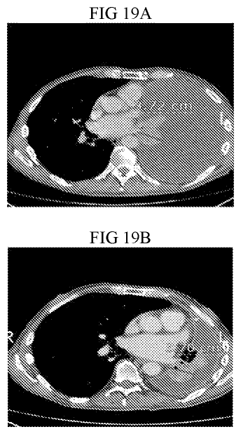

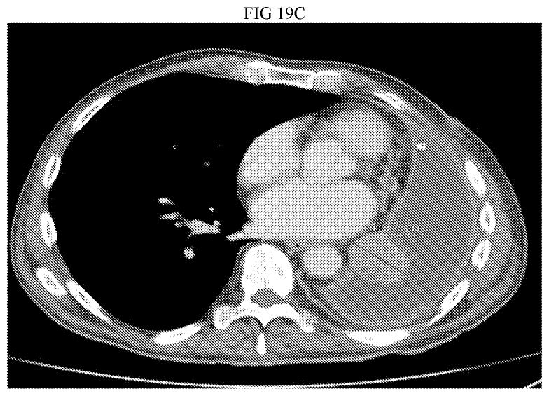

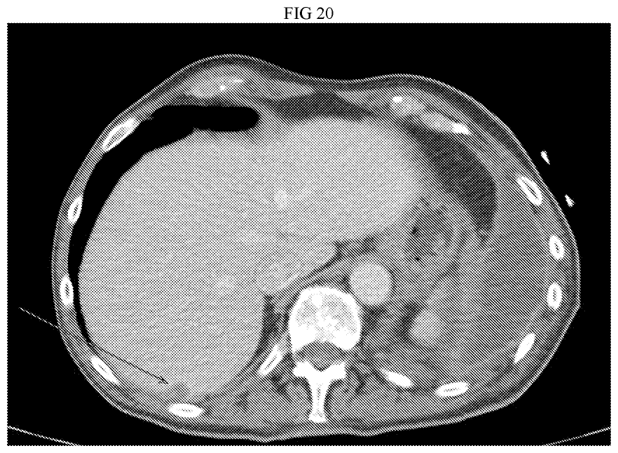

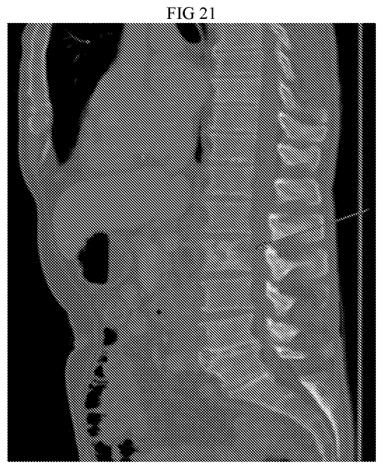

As shown in FIG. 17, Patient 3 showed stable disease through at least 6 weeks of treatment, with disease progression observed at the week 12 scan. FIG. 18 shows that Patient 3 experienced a reduction in pleural effusion drainage through 12 weeks of treatment. FIGS. 19A-19C are CT scans of Patient 3 at pre-treatment (19A), 6 weeks on treatment (19B), and 12 weeks on treatment (19C). FIGS. 20 and 21 are CT scans of Patient 3 taken at 12 weeks on treatment.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

We claim:

1. A method of treating cancer, comprising administering to a subject in need thereof thiostrepton in a single dose of about 50 mg to about 500 mg once per week, thereby treating the cancer.

2. The method of claim 1, wherein the single dose is from about 90 mg to about 450 mg.

3. The method of claim 2, wherein the single dose is selected from about 90 mg, about 120 mg, about 180 mg, about 270 mg, about 360 mg, and about 450 mg.

4. The method of any previous claim, wherein the single dose is administered to the subject once per week for at least 3 weeks.

5. The method of claim 4, wherein the single dose in one week is the same amount as the single dose administered in a different week.

6. The method of claim 4, wherein a first single dose is administered for 3 weeks, followed by administration of a second single dose for the next 3 weeks, wherein the first single dose and the second single dose are different.

7. The method of claim 6, wherein the second single dose is greater than the first single dose.

8. The method of claim 6, wherein the second single dose is less than the first single dose.

9. The method of any one of claims 1-8, wherein the thiostrepton is present in a pharmaceutical composition further comprising at least one pharmaceutically acceptable carrier.

10. The method of any one of claims 1-9, wherein the thiostrepton is administered locally.

11. The method of any one of claims 1-10, wherein the thiostrepton is administered intraperitnoeally, intrapleurally, subcutaneously, or intratumorally.

12. The method of any one of claims 1-11, wherein the thiostrepton is administered by a catheter, a tube, or a needle.

13. The method of any one of claims 1-10, wherein the thiostrepton is administered by an indwelling intraperitoneal catheter (IPC).

14. The method of any one of claims 1-10, wherein the cancer is selected from lung, breast, prostate, melanoma, esophageal, leukemia, cervical, liver, colon, gastric, colorectal, glioblastoma, head and neck, pancreatic, mesothelioma, and ovarian.

15. The method of any one of claims 1-10, wherein the cancer is malignant mesothelioma.

16. The method of claim 15, wherein the cancer is malignant pleural mesothelioma.

17. The method of claim 16, wherein the cancer is malignant epitheloid pleural mesothelioma.

18. The method of claim 16, wherein the cancer is malignant peritoneal mesothelioma.

19. The method of claim 14, wherein the cancer is lung cancer.

20. The method of claim 19, wherein the lung cancer is an adenocarcinoma.

21. The method of any one of claims 14-20, wherein the cancer is metastatic.

22. The method of any preceding claim, wherein the subject experiences stable disease for at least 12 weeks after administration of a first dose of thiostrepton.

23. The method of any preceding claim, wherein the subject experiences stable disease for at least 18 weeks after administration of a first dose of thiostrepton.

24. The method of any of claims 1-21, wherein the subject experiences stable disease for at least 24 weeks after administration of a first dose of thiostrepton.

25. The method of any preceding claim, wherein the subject experiences progression free survival for at least 12 weeks after administration of a first dose of thiostrepton.

26. The method of any preceding claim, wherein the subject experiences progression free survival for at least 18 weeks after administration of a first dose of thiostrepton.

27. The method of any of claims 1-21, wherein the subject experiences progression free survival for at least 24 weeks after administration of a first dose of thiostrepton.

28. The method of any of claims 1-21, wherein the subject experiences partial response for at least 12 weeks after administration of a first dose of thiostrepton.

29. The method of any of claims 1-21, wherein the subject experiences partial response for at least 18 weeks after administration of a first dose of thiostrepton.

30. The method of any of claims 1-21, wherein the subject experiences partial response for at least 24 weeks after administration of a first dose of thiostrepton.

31. The method of any one of claims 28-30, wherein the partial response comprises a reduction in tumor size of at least 10% relative to the tumor size before administration of thiostrepton.

32. The method of any one of claims 28-31, wherein the partial response comprises a reduction in tumor size of at least 30% relative to the tumor size before administration of thiostrepton.

33. The method of any one of claims 28-32, wherein the partial response comprises a reduction in tumor size of at least 60% relative to the tumor size before administration of thiostrepton.

34. The method of any preceding claim, wherein the subject experiences pleural effusion.

35. The method of claim 34, wherein the volume of pleural effusion is reduced 10 weeks after administration of a first does of thiostrepton relative to the volume of pleural effusion before administration of thiostrepton.

36. The method of claim 34, wherein the volume of pleural effusion is reduced 5 weeks after administration of a first does of thiostrepton relative to the volume of pleural effusion before administration of thiostrepton.

37. The method of claim 34, wherein the volume of pleural effusion is reduced 1 week after administration of a first does of thiostrepton relative to the volume of pleural effusion before administration of thiostrepton.

38. The method of any one of claims 35-37, wherein the volume of pleural effusion is about 30% less than the volume of pleural effusion before administration of thiostrepton.

39. The method of any one of claims 35-37, wherein the volume of pleural effusion is about 60% less than the volume of pleural effusion before administration of thiostrepton.

40. The method of any one of claims 35-37, wherein the volume of pleural effusion is about 90% less than the volume of pleural effusion before administration of thiostrepton.

41. A pharmaceutical composition comprising thiostrepton and at least one pharmaceutically acceptable carrier, wherein the pharmaceutical composition comprises from about 50 mg to about 500 mg of thiostrepton.

42. The pharmaceutical composition of claim 41, wherein the composition comprises from about 90 mg to about 450 mg of thiostrepton.

43. The pharmaceutical composition of claim 42, wherein the composition comprises about 90 mg, about 180 mg, about 270 mg, about 360 mg, or about 450 mg of thiostrepton.
